(19) United States
(12) Patent Application Publication
(10) Pub. No.: US 2006/0084585 A1
(43) Pub. Date: Apr. 20, 2006

(54) Peel-off nail polish without polish-remover

(76) Inventor: I-Ming Lin, Tainan City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/967,244

(22) Filed: Oct. 19, 2004

(51) Int. Cl. A61K 8/00

(52) U.S. Cl. 510/118

(57) ABSTRACT

Peel-off nail polish is composed of between 20% to about 30% by weight of Polyacryate, between 8% to about 18% by weight of Plasticiser, between 0.5% to about 5% by weight of Anti-setting agent, and between 60% to about 70% by weight of Solvent. The nail polish can be peeled off easily after drying without the necessity of using nail polish-remover.

### FIELD OF THE INVENTION

This invention relates to nail polish, and more particularly to nail polish being able to peel off without the necessity of using nail polish-remover.

### BACKGROUND OF THE INVENTION

Conventional nail polish currently on the market requires polish-remover to remove the polish from nail. The polish-remover is to solve the color layer by layer. In some occasions, the color may remain on the nail. Further, the polish-remover has a strong smell.

### SUMMARY OF THE INVENTION

It is the primary objective of the present invention to provide peel-off nail polish, which utilizes Polyacryate as the primary composition of the nail polish. The nail polish can be peeled off easily after drying.

It is another objective of the present invention to provide peel-off nail polish, which is easy to clean.

It is another objective of the present invention to provide peel-off nail polish, which saves time from peeling off the nail polish.

### DETAILED DESCRIPTION OF THE PREFERRED EMBODIMENT

The present invention comprises between 20% to about 30% by weight of Polyacryate, between 8% to about 18% by weight of Plasticiser, between 0.5% to about 5% by weight of Anti-setting agent, and between 60% to about 70% by weight of Solvent.

The Polyacryate is selected from Methyl, Methacrylate or Butyl Acrylate. The Plasticiser is selected from Dibutyl Phthalate or Dinonyl Phthalate. The Anti-setting agent is selected from Amine compound or Amide compound. The Solvent is selected from Ethyl Acetate, Butyl Acetate or Isobutanol.

The compound may be added with between 1% to about 10% by weight of color ingredients and appropriate additives. The color ingredients are selected from listing approved by Food and Drug Administration (FDA).

To apply the nail polish, it is easy to peel off the nail polish after drying without the necessity of using nail polish-remover.

I claim:

1. Peel-off nail polish comprising between 20% to about 30% by weight of Polyacryate, between 8% to about 18% by weight of Plasticiser, between 0.5% to about 5% by weight of Anti-setting agent, and between 60% to about 70% by weight of Solvent;
said Polyacryate selected from Methyl, Methacrylate or Butyl Acrylate, said Plasticiser selected from Dibutyl Phthalate or Dinonyl Phthalate, said Anti-setting agent selected from Amine compound or Amide compound, and said Solvent selected from Ethyl Acetate, Butyl Acetate or Isobutanol.

2. The peel-off nail polish, as recited in claim 1, wherein said peel-off polish further comprises between 1% to about 10% by weight of color ingredients and additives.
